(19) United States
(12) Patent Application Publication
(10) Pub. No.: US 2009/0241246 A1
(43) Pub. Date: Oct. 1, 2009

(54) SWIMMING GOGGLES HAVING AIR CHANNELS FOR COMFORT WEARING

(76) Inventors: Joo Dong Eun, Seoul (KR); Tae Soo Lee, Gyeonggi-Do (KR); Dong Chul Kim, Gyeonggi-Do (KR); Soo An Park, Gyeonggi-Do (KR)

Correspondence Address: GWIPS;Peter T. Kwon, Gwacheon P.O. Box 72, 119 Byeolyang Ro, Gwacheon City, Gyeonggi-Do, 427-600, KR

(21) Appl. No.: 12/310,439

(22) PCT Filed: Oct. 23, 2006
(86) PCT No.: PCT/KR2006/004324

§ 371 Date: Feb. 25, 2009

(30) Foreign Application Priority Data
Aug. 28, 2006 (KR) 10-2006-0081737

(51) Int. Cl. A61F 9/02

(52) U.S. Cl. 2/436

(57) ABSTRACT

A swimming goggle has improved to snugly fit around a user's eyes for comfort wearing. An air channel is adopted to remove an ocular pain due to a pressure in an inner space where the user's eyes are placed when the swimming goggles are worn. The swimming goggle comprises a pair of glasses disposed at a predetermined distance in front of the user's eyes, a set of looped cushions surrounding the glasses for directly contacting around the user's eyes, and a vent hole interconnecting an outer surface with an inner surface of the glasses or penetrating the looped cushions.

### BACKGROUND OF THE INVENTION

1. Field of the Invention

The present invention relates to a swimming goggle having air channels for comfort wearing. More particularly, the air channels for vent air and a pair of looped cushions around the glasses have adopted to snugly fit around the user's eyes to comfort wear by removing ocular pain due to the negative pressure in an area where the user's eyes are placed.

2. Related Prior Art

The swimming goggles are used to ensure a clear field of view and protect a user's eyes during swimming.

The swimming goggles include a lens part which is bent forming a predetermined inner space in front of the user's eyes.

Hereinafter, the conventional swimming goggles will be described with reference to FIG. 8. FIG. 8 shows conventional swimming goggles 10 in use. As shown in FIG. 8, the conventional swimming goggles 10 comprise a lens part 20, and a cushion member 12 disposed between a user's face and the lens part 20 in connection to the lens part 20.

To wear the swimming goggles 10, the user pushes the lens part 20 toward the face so that pressure of an inner space 14 becomes lower than the outer pressure, thereby generating a negative pressure in the inner space 14. Here, the cushion member 12 can also serve as a gasket that prevents passage of air through a gap between the inner space 14 and the outside.

The negative pressure of the inner space 14 usually causes pain to the user by drawing the user's eyes and the facial skin surrounding the eye pits to the swimming goggles 10. If the user wears the swimming goggles loosely so that the pressure of the inner space 14 does not drop much to relieve such pain, air-tightness of the swimming goggles 10 deteriorates and water may infiltrate the swimming goggles 10.

In addition, the negative pressure generated inside the conventional swimming goggles 10 thickens the eye lens slightly, thereby disturbing eyesight. Especially, professional swimming goggles for players are omitted with the cushion member 12 such as a silicon pad in order to reduce resistance. Therefore, when wearing the professional swimming goggles, the user would compress the lens part 10 more tightly. Accordingly, the negative pressure in the swimming goggles 10 increases, aggravating the disturbance of eyesight and even hindering a swimming competition.

### SUMMARY OF THE INVENTION

Therefore, the present invention has been made in view of the above-mentioned problems, and it is an object of the present invention to provide swimming goggles capable of preventing ocular pain caused by negative pressure of an inner space between the swimming goggles and a user's eyes.

It is another object of the present invention to provide swimming goggles capable of removing disturbance of the user's eyesight caused by the eye lens thickened due to pressure difference between the inside and the outside of the swimming goggles, so that the user can maintain normal eyesight while wearing the swimming goggles.

According to an aspect of the present invention, there is provided swimming goggles including a lens part disposed at a predetermined distance in front of a user's eyes and a cushion part surrounding the periphery of the lens part and directly contacting with the user's face, wherein a vent hole is formed to interconnect an outer surface with an inner surface of the lens part or penetrate the cushion part, thereby forming an air tunnel so that wearing comfort is enhanced.

The vent hole is formed on at least one position among one sides of an upper portion, a side portion, and a lower portion of the lens part, and one side of the cushion part.

In swimming goggles comprising a lens part disposed at a predetermined distance in front of a user's eyes and a cushion part surrounding the periphery of the lens part and directly contacting with the user's face, a vent hole may be formed interconnecting an outer surface with an inner surface of the lens part.

The vent hole may be formed at a lower portion of the lens part. In addition, the vent hole may be bent in the zigzag form.

According to another embodiment of the present invention, the vent hole comprises an inner vent hole formed on an inner surface at one side of the lens part; an extension hole extended from the inner vent hole and formed along an inside of the periphery of the lens part; and an outer vent hole extended from the extension hole and formed on an outer surface of the other side of the lens part.

According to yet another embodiment of the present invention, the vent hole comprises a first outer hole formed on an upper outer surface of the lens part; a first extension hole extended from the first outer hole up to the middle of the lens part, along an inside of the periphery of the lens part; a first inner hole extended from the first extension hole and formed on an inner surface of the lens part; a first valve connected between the first extension hole and the first inner hole and capable of opening toward the first inner hole; a second outer hole formed on a lower outer surface of the lens part; a second extension hole extended from the second outer hole up to the middle of the lens part, along an inside of the periphery of the lens part; a second inner hole extended from the second extension hole and formed on an inner surface of the lens part; and a second valve connected between the second extension hole and the second inner hole and capable of opening toward the second inner hole.

The cushion part includes a concave part formed along the middle of a surface thereof to improve close contact between the surface and the user's face. The vent hole has diameter not greater than 3 mm to prevent infiltration of a water drop. The vent hole has diameter not greater than 1 mm not to allow infiltration of a water drop.

As described above, swimming goggles according to the present invention are provided with vent holes connecting an outer surface and an inner surface of a lens part or penetrating a cushion part, so that pressure of an inner space of the swimming goggles is maintained equal to atmospheric pressure. Accordingly, wearing comfort can be enhanced. In addition, infiltration of water can be effectively prevented by the vent holes disposed at random positions such as upper, side and lower portions of the lens part or the cushion part, as long as not disturbing a user's eyesight.

When forming the vent holes in a zigzag form, infiltration of water can be prevented more effectively.

According to an embodiment of the present invention, an inner vent hole, an extension hole extended from the inner vent hole, and an outer vent hole extended from the extension hole are provided so that an air tunnel is elongated. Consequently, wearing comfort can be enhanced while preventing infiltration of water more effectively.

According to another embodiment of the present invention, inner pressure of the swimming goggles can be maintained equally to the outer pressure even during a short time of entering and exiting the water, by a multiple valve structure comprising first and second outer holes, first and second extension holes, first and second inner holes, and first and second valves.

Additionally, the cushion part having a concave part formed in the middle of a surface thereof directly contacting with the user's face can enhance close contact between the swimming goggles and the user's face.

The vent holes have diameter not greater than 3 mm to prevent infiltration of a water drop.

When the vent holes are not greater than 1 mm in diameter, even a micro water drop can be prevented from infiltrating.

In the swimming goggles having a valve form, diameter of the vent holes and diameter of a valve mounting part may be different from each other. When the valve mounting part has greater diameter than the vent hole, mounting of the valve becomes easier.

Moreover, according to the present invention, disturbance of eyesight caused when the eye lens are thickened by pressure difference between the inside and the outside of the conventional swimming goggles can be solved. Therefore, the user can swim with normal eyesight.

Especially, professional swimmers can improve their swimming record by maintaining the normal eyesight during competition.

### BRIEF DESCRIPTION OF THE DRAWINGS

FIG. 1 is a sectional view showing main elements of swimming goggles according to a first embodiment of the present invention;

FIG. 2 shows the swimming goggles according to the first embodiment, in use;

FIG. 3 is a sectional view showing main elements of swimming goggles according to a second embodiment of the present invention;

FIG. 4 is a front view of swimming goggles according to a third embodiment of the present invention;

FIG. 5 is a front sectional view of swimming goggles according to a fourth embodiment of the present invention, with a first valve closed;

FIG. 6 is a front sectional view of the swimming goggles according to the fourth embodiment, with a second valve closed;

FIG. 7 is a sectional view of swimming goggles according to a fifth embodiment of the present invention.

FIG. 8 shows conventional swimming goggles in use.

### DESCRIPTION OF THE PREFERRED EMBODIMENTS

Description will now be made in detail to certain embodiments of the present invention with reference to the accompanying drawings.

Well-known functions and constructions are not described in detail since they would obscure the invention in unnecessary detail. Terms described herein are determined in consideration of functions in this invention, and definition of the terms may vary according to a producer's intention or custom. Also, the definition of the terms is determined based on the contents throughout the specifications.

### First Embodiment

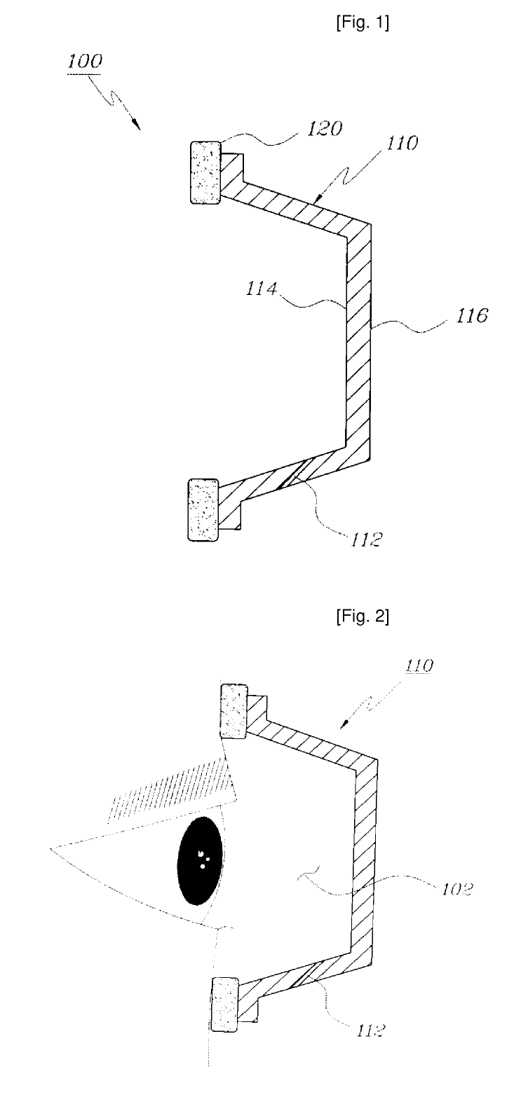

Swimming goggles having enhanced wearing comfort according to a first embodiment of the present invention will be described with reference to FIGS. 1 and 2. FIG. 1 is a sectional view showing the main elements of the swimming goggles according to the first embodiment, and FIG. 2 is a view showing the swimming goggles according to the first embodiment in use.

Hereinafter, the swimming goggles having enhanced wearing comfort will be referred to simply as ‘swimming goggles’ without particular explanation. As shown in FIG. 1, the swimming goggles 100 include a vent hole 112 formed at a lower portion of a lens part 110 and interconnecting an inner surface 114 and an outer surface 116 of the lens part 110. A cushion part 120 is formed to fit the swimming goggles 100 on a user's facial skin curve and protect the user from impacts. The cushion part 120 can be made of silicon or foamed synthetic resin.

Although the vent hole 112 is formed at a lower part of the lens part 110 according to the drawings, the present invention is not limited so. As long as not interfering with the user's visual field, the vent hole 112 can be disposed at any positions such as an upper portion and a side portion.

Although the vent hole 112 is formed at the lens part 110 in this embodiment, the present invention is not limited so. The vent hole 112 may be formed through the cushion part 120 of the swimming goggles 100.

The vent hole 112 of the cushion part 120 can be disposed on any position of the cushion part 120. More specifically, the vent hole 112 may be formed at only the lens part 110, only the cushion part 120, or both the lens part 110 and the cushion part 120. As necessitated, one or more vent holes 112 can be formed in predetermined arrangement.

According to an exemplary embodiment of the present invention, diameter of the vent hole 112 is not greater than 3 mm. As generally known, a water drop is equal to or greater than 1 mm in diameter. Especially, a water drop splashed in a swimming pool is usually larger than a sprayed water drop, that is, not less than 3 mm in diameter. Therefore, diameter of the vent hole 112 can be set equal to or less than 3 mm.

Considering that certain water drops have diameter not greater than 1 mm, the vent hole 112 preferably has diameter not greater than 1 mm in order to prevent water infiltration more effectively.

Accordingly, the vent hole 112 according to the embodiment of the present invention has smaller diameter than a water drop, so as not to allow a water drop to pass therethrough while allowing steam particulates having diameter of several to tens of micrometers. Therefore, while water cannot infiltrate from the outside to an inner space between the user's eye and the lens part 110, air and steam particulates of the inner space can pass freely through the vent hole 112.

Even when water surges in through the vent hole 112, infiltration of the water can be prevented by the air in the swimming goggles 100. This is the same principle in that water does not infiltrate an opening of a bottle which is put into water upside down.

When wearing the swimming goggles 100 according to the first embodiment of the present invention, as shown in FIG. 2, ocular pain by pressure difference can be overcome since the air can freely pass between the inner space and the outside through the vent hole 112.

### Second Embodiment

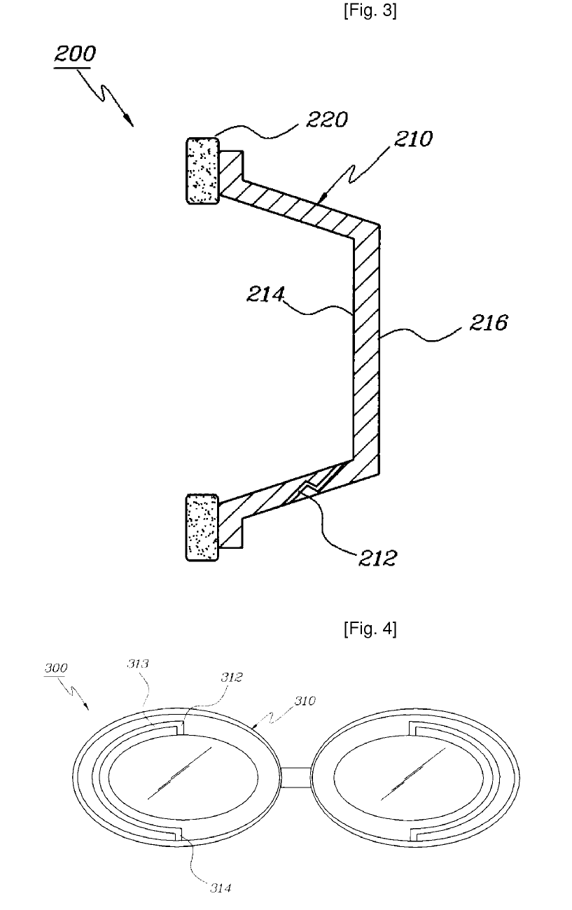

Swimming goggles according to a second embodiment of the present invention will now be described with reference to FIG. 3.

FIG. 3 is a sectional view showing main elements of the swimming goggles according to the second embodiment.

Referring to FIG. 3, swimming goggles 200 according to the second embodiment of the present invention is structured substantially in the same manner as the swimming goggles 100 of the first embodiment, except that a vent hole 212 of the swimming goggles 200 has a zigzag form.

When the vent hole 212 is in the zigzag form, as shown in the second embodiment, water infiltration can be more effectively prevented while the air can still smoothly pass.

As well as in the first embodiment, although the vent hole 212 is disposed at a lower portion of a lens part 210 in the second embodiment, the vent hole 212 can be disposed on any other positions such as upper and side portions of the lens part 210. It will be sure understood that the vent hole 212 can be disposed on any position of a cushion part 220.

### Third Embodiment

Hereinafter, swimming goggles according to a third embodiment of the present invention will be described with reference to FIG. 4.

FIG. 4 is a front view of swimming goggles 300 according to the third embodiment of the present invention.

As shown in FIG. 4, the swimming goggles 300 of the third embodiment have an inner vent hole 312, an extension hole 313, and an outer vent hole 314. Except this, the third embodiment has substantially the same structure as the first embodiment.

The inner vent hole 312 is disposed on an upper inner surface of a lens part 310. However, position of the inner vent hole 312 is not limited to the upper portion of the lens part 310, but can be a side portion or a lower portion.

The extension hole 313 is formed along an inside of the periphery of the lens part 310, being extended from the inner vent hole 312.

The outer vent hole 314 can be disposed on any position of an outer side of the lens part 310. However, in order to effectively prevent infiltration of water, the outer vent hole 314 is preferred to be disposed at the lower portion.

According to the above configuration, the swimming goggles 300 according to the third embodiment of the present invention are capable of preventing infiltration of water almost completely.

The vent holes 312, 313 and 314 in the third embodiment can also be formed at a cushion part.

### Fourth Embodiment

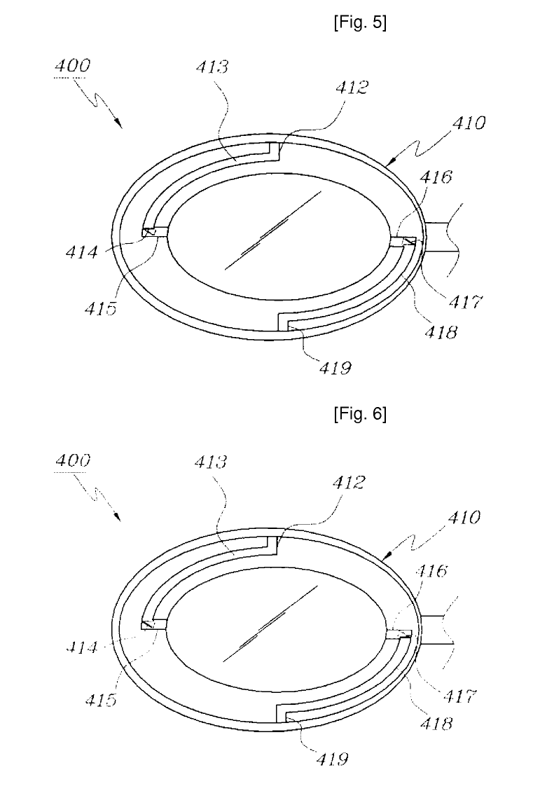

Hereinafter, a fourth embodiment of the present invention will be described with reference to FIGS. 5 and 6.

FIG. 5 is a front sectional view of swimming goggles according to the fourth embodiment, with a first valve closed. FIG. 6 is a front sectional view of swimming goggles according to the fourth embodiment, with a second valve closed.

As shown in the drawings, in swimming goggles 400 according to the fourth embodiment of the present invention, vent holes are divided into first and second outer holes 412 and 419, first and second extension holes 413 and 418, first and second inner holes 415 and 416, and first and second valves 414 and 417.

The first outer hole 412 is formed on an upper outer surface of a lens part 410. The first extension hole 413 is formed along an inside of the periphery of the lens part 410, being extended from the first outer hole 412 up to the middle of the lens part 410.

The inner hole 415 is formed on an inner surface of the lens part 410, being extended from the first extension hole 412.

The first valve 414 is connected between the first extension hole 413 and the first inner hole 412. Additionally, the first valve 414 can open toward the first inner hole 415.

The second outer hole 419 is formed on a lower outer surface of the lens part 410.

The second extension hole 418 is formed along an inside of the periphery of the lens part 410, being extended from the second outer hole 419.

The second inner hole 416 is formed on an inner surface of the lens part 410, being extended from the second extension hole 418.

The second valve 417 is connected between the second extension hole 418 and the second inner hole 416, and capable of opening toward the second inner hole 416.

The operation of the vent hole according to the fourth embodiment of the present invention will now be described. As the user enters water wearing the swimming goggles 400, the water surges to the second outer hole 419 and accordingly, the second valve 417 is opened. The first valve 414 is closed simultaneously. As a result, constant pressure can be maintained in an inner space of the swimming goggles 400, as shown in FIG. 5.

When the user exits from the water wearing the swimming goggles 400, the water surges to the first outer hole 412 and the first valve 414 is opened. Simultaneously, the second valve 417 is closed. As a result, constant pressure can be maintained in the inner space of the swimming goggles 400. This state is shown in FIG. 6.

As described above, inner pressure of the swimming goggles 400 can be constantly maintained by the first and the second valve 414 and 417 being selectively opened and closed, thereby improving the wearing comfort.

The vent holes in the embodiments of the present invention may have approximately 1 mm diameter. Diameter of a valve mounting part may be approximately 3 mm for smooth mounting of the valve.

However, the present invention is not limited to the embodiments. Therefore, for example, both the vent holes and the valve mounting parts can be equal to or less than 3 mm in diameter. Furthermore, for more complete prevention of water infiltration, both the vent holes and the valve mounting parts may be equal to or less than 1 mm in diameter for more complete prevention of water infiltration.

### Fifth Embodiment

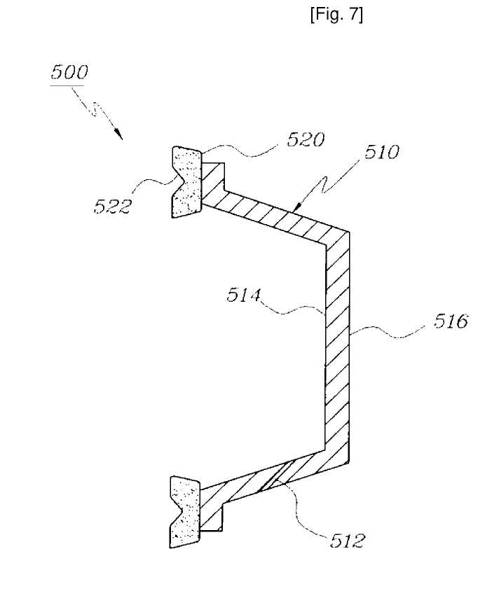

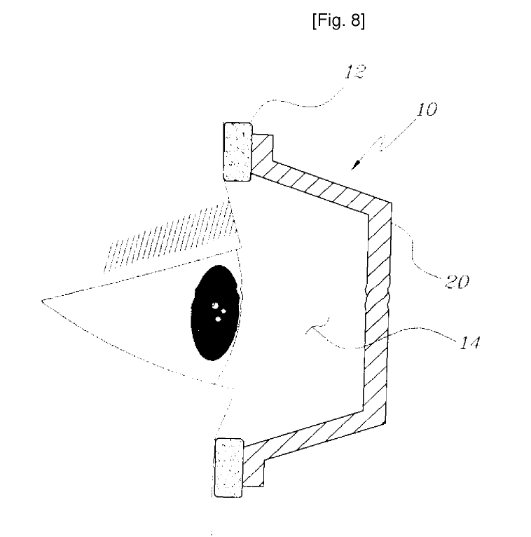

Swimming goggles according to a fifth embodiment of the present invention will be described hereinafter, with reference to FIG. 7.

FIG. 7 is a sectional view showing main elements of swimming goggles 500 according to the fifth embodiment of the present invention.

Referring to FIG. 7, in comparison with the swimming goggles 100 of the first embodiment, the swimming goggles 500 of the fifth embodiment further comprise a concave part 522 formed on a cushion part 520 along the middle of a surface of the cushion part 520, directly contacting with the user's face. The other structures of the fifth embodiment are substantially the same as in the first embodiment.

The above-structured cushion part 520 of the swimming goggles 500 according to the fifth embodiment can enhance the wearing comfort since the concave part 522 is transformed and closely contacted with the user's face when the user wears the swimming goggles 500.

While the invention has been shown and described with reference to certain embodiments thereof, it will be understood by those skilled in the art that various changes in form and details may be made therein without departing from the spirit and scope of the invention as defined by the appended claims.

1-8. (canceled)

9. A swimming goggle equipped with air channels to snugly fit around eyes, said swimming goggle comprising a pair of glasses and looped cushions disposed periphery of the glasses to directly contact around a user's eyes, wherein a vent hole is formed in such a manner that the vent hole interconnects an outer surface with an inner surface of the glasses or penetrates the cushions, or the vent hole is formed at the glasses and the cushions in such a manner that the vent hole penetrates from the outer surface of the glasses to the inner surface of the glasses and the cushions.

10. The swimming goggle of claim 9, wherein said vent hole comprises:
an inner vent hole formed on an inner surface at one side of the glasses;
an extension hole, which is extended from the inner vent hole and is formed along an inside of the glasses while being spaced a predetermined interval from the periphery of the glasses; and
an outer vent hole, which is extended from the extension hole and is formed on an outer surface of another side of the glasses.

11. The swimming goggle of claim 9, wherein the vent hole comprises:
a first outer hole formed on an upper outer surface of the glasses;
a first extension hole extended from the first outer hole to a middle of the glasses along an inside of the glasses while being spaced a predetermined interval from the periphery of the glasses;
a first inner hole, which is extended from the first extension hole and is formed on the inner surface of the glasses;
a first valve, which is connected between the first extension hole and the first inner hole and can be opened toward the first inner hole;
a second outer hole formed on a lower outer surface of the glasses;
a second extension hole extended from the second outer hole to the middle of the glasses along the inside of the glasses while being spaced a predetermined interval from the periphery of the glasses;
a second inner hole, which is extended from the second extension hole and is formed on the inner surface of the glasses; and
a second valve, which is connected between the second extension hole and the second inner hole and can be opened toward the second inner hole.

12. The swimming goggle of claim 9, wherein the cushions includes a concave part formed along the middle of a surface thereof to improve close contact between the surface and the user's face.

13. The swimming goggle of claim 9, wherein the vent hole has diameter not greater than 3 mm to prevent infiltration of a water drop.
